(19) United States
(12) Patent Application Publication
(10) Pub. No.: US 2016/0238579 A1
(43) Pub. Date: Aug. 18, 2016

(54) DETECTING SYSTEM AND DETECTING METHOD FOR AIR QUALITY BASED ON A ROBOT

(71) Applicant: YUTOU TECHNOLOGY (HANGZHOU) CO., LTD., Hangzhou City, Zhejiang (CN)

(72) Inventors: Mingjun CAI, Zhejiang (CN); Xin LIU, Zhejiang (CN)

(21) Appl. No.: 14/761,871

(22) PCT Filed: Jun. 12, 2015
(86) PCT No.: PCT/CN2015/081401

§ 371 Date: Jul. 17, 2015

(30) Foreign Application Priority Data
Jan. 12, 2015 (CN) 201510015906.5

(51) Int. Cl. G01N 33/00; F24F 11/00

(52) U.S. Cl. CPC G01N 33/0062; G01N 33/004; F24F 11/0012; F24F 11/0015; F24F 11/0017; F24F 11/006; G01N 2033/0068; F24F 2011/0026; F24F 2011/0061

(57) ABSTRACT

A detecting system for air quality based on a robot is described. The system comprises a collection unit, a processing unit, a storage unit, an execution unit, and an input/output device. The detection method comprises: controlling the collection unit to detect the air's status within the environment of the robot, and to form an output of collection data; controlling the processing unit to receive the collection data and receive the storage data output by the storage unit simultaneously, match the storage data to the collection data, and output a processing result formed by the collection data and the storage data; controlling the input/output device to receive the processing result and output a corresponding processing result. The benefit of the present disclosure compared to prior art is that this invention makes it more convenient for the user to see the air quality information of their present environment.

### BACKGROUND OF THE INVENTION

1. Field of the Invention

The present disclosure relates to the field of robotics, more specifically, to a detecting system and a method for air quality based on a robot.

2. Description of the Related Art

Nowadays the environment changes rapidly, and air quality fluctuates according to plant emissions, automobile exhaust, and exhaust gas. Increased air pollution is linked to an escalation in the incidences of diseases, such as asthma, lung cancer, cardiovascular disease, birth defects, and premature death. It is important to monitor the air quality in our living and working environments, and to maintain purified air. Although air detecting devices and air detecting systems have been provided in prior art, those air detecting device and the air detecting system can only be used to detect a single air component, for example, it can only detect the concentration of carbon dioxide, or it can only detect the concentration of another specific harmful gas present in the air. Therefore, because the detecting method is particular, it cannot present the status of the present physical environment fully. Meanwhile, neither the air detecting devices nor the air detecting systems can execute necessary operations, such as purifying the air, which is relative to the present environment, so that the present environment is consistent.

### SUMMARY OF THE INVENTION

To resolve the shortcoming of the prior art, the invention provides a detecting system as well as a detection method for air quality based on a robot, which adjusts the status of air with various detecting types.

The technical scheme of the invention is:

A detecting system for air quality based on a robot, comprising:

a collection unit configured in a preset position on a robot, which is used to detect the air's condition in immediate surroundings of the robot, and then to form an output of collection data;

a processing unit connected to the collection unit, which is configured to receive the collection data and simultaneously receive the storage data which is output by a storage unit and is matched to the collection data, and then to form a processing result in accordance with the collection data and the storage data, as well as to simultaneously output a control signal;

a storage unit connected to the processing unit, which is configured to store the processing result;

an execution unit, whose control end is connected to the processing unit by a communication module to execute, under the effect of the control signal, actions that match the control signal;

an input/output device connected to the processing unit, which is used to receive and output the processing result, or to receive control instructions input by users, and then to form a control signal and transmit it to the processing unit.

Preferably, the collection unit comprises a temperature sensor, which is configured to collect the temperature information of the immediate surroundings of the robot, and then to form an output of temperature signals.

Preferably, the collection unit comprises a humidity sensor, which is configured to collect the humidity information of the immediate surroundings of the robot, and then to form an output of humidity signals.

Preferably, the collection unit comprises a carbon dioxide sensor, which is configured to collect the carbon dioxide concentration information of the immediate surroundings of the robot, and then to form an output of carbon dioxide concentration signals.

Preferably, the collection unit comprises a dust sensor, which is configured to collect the dust concentration information of the immediate surroundings of the robot, and then to form an output of dust concentration signals.

Preferably, the collection unit comprises a gas sensor, which is configured to collect the control gas composition, concentration information of the immediate surroundings of the robot, and then to form an output of gas signals.

A detecting method for air quality based on a robot, wherein it comprises of the following steps:

(a) controlling a collection unit to detect the air's condition in the immediate surroundings of the robot, and to form an output of collection data;

(b) controlling a processing unit to receive the collection data and simultaneously receive the storage data that is output by a storage unit and is matched to the collection data, and to form a processing result in accordance with the collection data and the storage data;

(c) controlling an input/output device to receive the processing result and output the corresponding processing result.

Preferably, it further comprises the following steps:

(d) controlling the input/output device to receive control instructions input by the user, and to form a control signal and transmit it to the processing unit;

(e) making the processing unit to receive the control signal, and then forming the processing result in accordance with the control signal, the processing result is output; and execute step (c).

The benefit of the invention comparative to the prior art is, by adopting the above technical solution, is that it is more convenient for the user to know the air's status information of present environment in real time, and easier to make an necessary adjustments which is beneficial to the health of the user.

### BRIEF DESCRIPTIONS OF THE DRAWINGS

FIG. 1 shows a structure diagram;

FIG. 2 shows a process schematic diagram.

### DETAILED DESCRIPTIONS

Hereinafter, certain exemplary embodiments, according to the present invention, will be described with reference to the accompanying drawings. This does not limit the scope of the invention.

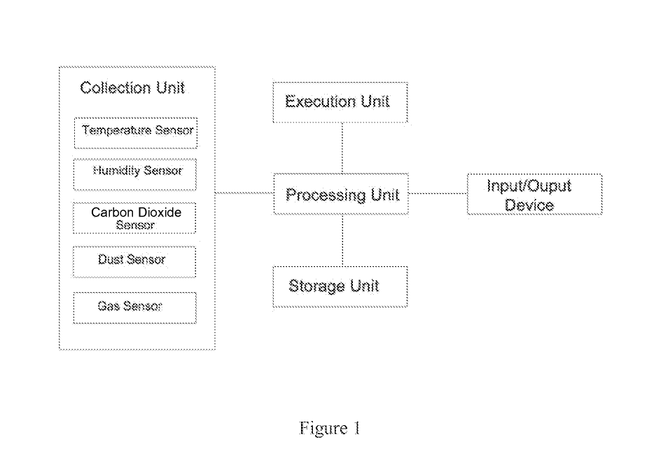

Referring to FIG. 1, the detecting system for the air quality based on a robot comprises:

a collection unit configured in a preset position on a robot, which is used to detect the air's condition in the immediate surroundings of the robot, and then to form an output of collection data;

a processing unit connected to the collection unit, which is configured to receive the collection data and simultaneously receive the storage data, which is output by a storage unit, which is matched to the collection data, as well as to form a processing result in accordance with the collection data and the storage data; a storage unit connected to the processing unit, configured to store the processing result;

an execution unit, whose control end is connected to the processing unit by a communication module to execute, under the effect of the control signal, actions that match the control signal; the execution unit can be a temperature adjustment unit, an air purification unit, a humidity adjustment unit, or an air exhaust adjustment unit;

an input/output device connected to the processing unit, which is used receive and output the processing result, as well as to receive control instructions input by the user, and to form a control signal and transmit it to the processing unit.

The working principle of the invention is the following: the collection unit collects information on the air's condition of the immediate surroundings of the robot, and then forms collection data and outputs it to the processing unit; the processing unit comprehensively analyzes and processes the collection data in accordance with the collection date and the existing processing result stored in the storage unit, which is used to form and output the processing result of the air conditions in the immediate surroundings; the input/output device can be a voice playing device and/or a display device configured to play and/or display the processing result; the execution unit executes the corresponding operations and improves the air's quality in the immediate surroundings. The input/output device can also receive the control instruction input by the user, and transform the control instruction to one that can be identified by the processing unit, under the effect of which, a processing result is formed and output to the output/input device, the output/input device then displays the processing result. By adopting the above technical solution, it facilitates the user to know the information regarding the air's condition within the immediate surroundings in real time, and it automatically makes necessary adjustments, which is beneficial to the health of the user.

In a further preferred embodiment, the collection unit comprises:

a temperature sensor, which is configured to collect the temperature information of the immediate surroundings of the robot, as well as to form an output of temperature signal. If the environment temperament is too low or too high, the processing unit sends out the control command, according to the present temperature signal, to adjust the temperature adjustment device, to maintain the temperature at a the level which people feel comfortable. The temperature adjustment device can further perform as an air conditioner, a receiver end of the air condition control unit is connected to the processing unit, the receiver end is configured to receive the remote control command sent from the processing unit, and the remote control commands are matched to the air condition; the control unit then executes the corresponding operation according to the remote control command, such as sending hot airflow or sending cold airflow.

When the indoor temperature ascends continually and exceeds the threshold preset by the processing unit, the processing unit determines the heat source and sends a control signal to the sprinkler, the sprinkler is then initiated; the sprinkler then sprays liquid at the heat source to cool down it, so to avoid the fire hazard.

In a further preferred embodiment, the collection unit comprises a humidity sensor, which is configured to collect the humidity information of the immediate surroundings of the robot, and then to form an output of the humidity signal. The processing unit sends out the control command, according to the present humidity signal, to adjust the humidity adjustment device, which maintains the humidity at a level that makes people feel comfortable. The humidity adjustment device can further perform as a humidifier, a control end of the humidifier is connected to the processing unit, the control end outputs corresponding moisture under the effect of the control command, to adjust the indoor temperature. The humidity adjustment device can also be an air conditioner.

In a further preferred embodiment, the collection unit comprises a carbon dioxide sensor configured to collect the carbon dioxide concentration information of the immediate surroundings of the robot, it then form an output signal of the carbon dioxide concentration. The control unit sends out a control command according to the present carbon dioxide concentration signal, which is used to adjust ventilation, thus adjusting the present carbon dioxide concentration. The ventilation unit can be a ventilating fan. The control end of the ventilating fan is connected to the processing unit; the ventilating fan is initiated under the effect of the control command, so that the ventilation is accelerated, and the carbon dioxide concentration is decreased.

In a further preferred embodiment, the collection unit comprises a dust sensor, which is configured to collect the dust concentration information of the immediate surroundings of the robot, and then to form an output of the dust concentration signal. The control unit sends out control command according to the present dust concentration signal to adjust the air purification unit, which adjusts the dust concentration in the air. The air purification unit can further perform as an air cleaner. The control end of the air cleaner is connected to the processing unit; the air cleaner is initiated under the effect of the control command to purify the indoor air, and to improve the indoor air quality. The purification unit can also be formed by an exhaust system of an air condition.

In a further preferred embodiment, the collection unit comprises a gas sensor, which is configured to collect the control gas composition and concentration information of the immediate surroundings of the robot, and then it forms an output of the gas signal. The gas sensor is mainly used for detecting any harmful gas concentration in the air. The processing unit sends out control commands, according to the present gas composition and concentration information, which is used to adjust the air purification unit, then, in turn adjusts the harmful gas in the air. The harmful gas includes but is not limited to methanal, volatile organic compounds, etc. The air purification unit can further perform as an air cleaner.

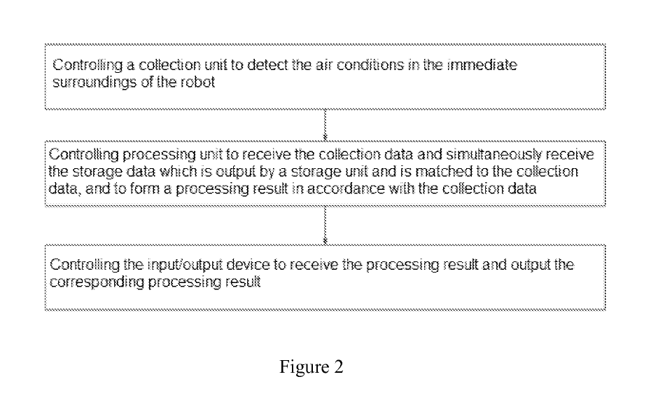

Referring to FIG. 2, a detecting method for air quality, based on the robot, comprises the following steps:

(a) controlling a collection unit to detect the air conditions in the immediate surroundings of the robot, and then to form an output of collection data;

(b) controlling processing unit to receive the collection data and simultaneously receive the storage data which is output by a storage unit and is matched to the collection data, and to form a processing result in accordance with the collection data;

(c) controlling the input/output device to receive the processing result and output the corresponding processing result.

In a further preferred embodiment, it also comprises the following steps:

(d) controlling the input/output device to receive the control instructions input by the user, and to form a control signal and transmit it to the processing unit;

(e) making the processing unit receive the control signal, and then forming the processing result in accordance with the control signal, the processing result is output; and execute step (c).

The working principle of the detection method for the air's quality, based on robot, has been described in the above detection system clearly, and it will not be described any more.

The above description is only directed to some preferred embodiments of the invention, and it does not limit the implement method and protecting scope of the invention. It is obvious for those skilled in the art that the changes and variations made by the specification and drawings of the invention should fall within the scope of the invention.

What is claimed is:

1. A detecting system for air quality based on a robot, comprising:
a collection unit configured in a preset position on a robot, which is used to detect an air condition in immediate surroundings of the robot, and then to form an output of collection data;
a processing unit connected to the collection unit, which is configured to receive the collection data and simultaneously receive storage data which is output by a storage unit, match the storage data to the collection data, and then form a processing result in accordance with the collection data and the storage data, as well as to simultaneously output a control signal;
the storage unit connected to the processing unit, and which is configured to store the processing result;
an execution unit, whose control end is connected to the processing unit by a communication module to execute, under an effect of the control signal, actions that match the control signal;
an input/output device connected to the processing unit, which is used to receive and output the processing result; or to receive control instructions input by users and then to form the control signal and transmit the control signal to the processing unit.

2. The detecting system for air quality based on the robot as claimed in claim 1, wherein the collection unit comprises a temperature sensor, which is configured to collect temperature information of the immediate surroundings of the robot, and then to form an output of temperature signals.

3. The detecting system for air quality based on the robot as claimed in claim 1, wherein the collection unit comprises a humidity sensor, which is configured to collect humidity information of the immediate surroundings of the robot, and then to form an output of humidity signals.

4. The detecting system for air quality based on the robot as claimed in claim 1, wherein the collection unit comprises a carbon dioxide sensor, which is configured to collect carbon dioxide concentration information of the immediate surroundings of the robot, and then to form an output of carbon dioxide concentration signals.

5. The detecting system for air quality based on the robot as claimed in claim 1, wherein the collection unit comprises a dust sensor, which is configured to collect dust concentration information of the immediate surroundings of the robot, and then to form an output of dust concentration signals.

6. The detecting system for air quality based on the robot as claimed in claim 1, wherein the collection unit comprises a gas sensor, which is configured to collect gas composition and concentration information of the immediate surroundings of the robot, and then to form an output of gas signals.

7. A detecting method for air quality based on a robot comprising:
(a) controlling a collection unit to detect an air condition in the immediate surroundings of the robot, and form an output of collection data;
(b) controlling a processing unit to receive the collection data and simultaneously receive storage data that is output by a storage unit, match the storage data to the collection data, and form a processing result in accordance with the collection data and the storage data;
(c) controlling an input/output device to receive the processing result and output a corresponding processing result.

8. The detecting method for air quality based on the robot as claimed in claim 7, further comprising:
(d) controlling the input/output device to receive control instructions input by the user, form a control signal, and transmit the control signal to the processing unit;
(e) making the processing unit receive the control signal, the processing result then being formed in accordance with the control signal and then output; and executing step (c).
